(19) United States
(12) Patent Application Publication
(10) Pub. No.: US 2018/0328654 A1
(43) Pub. Date: Nov. 15, 2018

(54) METHODS FOR RECOVERING ALKENES AND NITROGEN FROM PROCESS GAS STREAMS

(71) Applicant: Linde Aktiengesellschaft, Munich (DE)

(72) Inventors: Nicole RUMORE, Monmouth Junction, NJ (US); Naveed ASLAM, Houston, TX (US)

(21) Appl. No.: 15/591,880

(22) Filed: May 10, 2017

(51) Int. Cl. F25J 3/02; F25J 3/08; C07C 7/09

(52) U.S. Cl. CPC F25J 3/0238; F25J 3/0219; F25J 3/0257; F25J 3/08; F25J 2230/08; F25J 2200/76; F25J 2210/12; F25J 2215/62; F25J 2215/64; C07C 7/09

(57) ABSTRACT

Methods and systems for recovering alkenes (e.g. ethylene, propylene) and nitrogen from process gas streams, including multi-step condensing of the process gas stream, are provided herein.

### FIELD OF THE INVENTION

This invention relates to methods utilizing heat integration for recovering alkene and nitrogen from various process gas streams.

### BACKGROUND OF THE INVENTION

Valuable hydrocarbons, such as lower alkenes, may be present in a multitude of process gas streams at varying concentrations and typically amongst other components. For example, ethylene and/or propylene may be present in cracked gas streams from hydrocarbon crackers, which also may contain nitrogen, methane, ethane, propane, hydrogen, other higher hydrocarbons and other impurities. Additionally, during polymerization processes, unreacted ethylene or propylene monomer may be present in streams along with inert purge gases, such as nitrogen. Thus, it would be desirable to be able to recover such valuable components, such as alkenes and nitrogen, for reuse in the process. However, typically, such streams containing alkene process gas (e.g., ethylene, propylene) are removed from the system via flaring or with a thermal oxidizer. Prior to flaring or thermal oxidizing, recovery processes may be employed for recovering as much of the alkene as possible so it is not lost when flared or oxidized. Examples of existing recovery processes include compression and condensation systems as well as use of pressure swing adsorption systems and/or membranes. However, existing recovery processes may still be limited in the amount of alkene and nitrogen recovery, as well as impurity removal. Further processing may also be required at additional costs to recover further amounts of alkene and nitrogen. Moreover, process gas streams comprising alkene may have higher temperatures; thus, requiring additional energy and resources, such as liquid nitrogen, to sufficiently lower the temperature of the process gas stream such that it may be suitably utilized in a condensation system.

Therefore, a need remains for improved and more effective methods which utilize heat integration for recovery of alkenes and nitrogen, as well as removal of impurities from process gas streams.

### SUMMARY OF THE INVENTION

It has been found that impurities from process gas streams may be sufficiently removed and alkenes and nitrogen may be sufficiently recovered by performing a combination of process steps including cooling a process gas stream to remove a portion of the impurities via a condensate stream followed by further cooling of the process gas stream for alkene recovery. Furthermore, streams produced during the process may be advantageously recycled and utilized during the process and provide sufficient cooling of the process gas streams.

Thus, in one aspect, this disclosure relates to a method for recovering C₂-C₄ alkene and nitrogen from a process gas stream comprising: cooling the process gas stream comprising C₂-C₄ alkene in a cooling apparatus with a first gaseous cooling medium or by mechanical means under suitable conditions to produce a first cooled process gas stream; cooling the first cooled process gas stream in a first condenser vessel with a second cooling medium under suitable conditions to produce a first condensate comprising impurities, a second cooled process gas stream and a first gaseous nitrogen stream; cooling the second cooled process gas stream in a second condenser vessel with a third cooling medium under suitable conditions to produce a second condensate comprising C₂-C₄ alkene, a vent gas stream and a second gaseous nitrogen stream; and recycling at least one of the following: (i) at least a portion of the first gaseous nitrogen stream and/or the second gaseous nitrogen stream to the cooling apparatus for use as at least a portion of the first gaseous cooling medium; or (ii) at least a portion of the second gaseous nitrogen stream to the first condenser vessel for use as at least a portion of the second cooling medium.

In still another aspect, this disclosure relates to a system for recovering C₂-C₄ alkene from a process gas stream comprising: a process gas stream comprising C₂-C₄ alkene; optionally, a first gaseous cooling medium stream; a first cooled process gas stream; a second cooling medium stream; a first condensate stream comprising impurities; a first gaseous nitrogen stream; a second cooled process gas stream; a third cooling medium stream; a second condensate stream comprising C₂-C₄ alkene; a vent gas stream; and a second gaseous nitrogen stream, wherein at least one of the following is present: (i) the first gaseous cooling medium stream, if present, comprises at least a portion of the first gaseous nitrogen stream and/or the second gaseous nitrogen stream; or (ii) the second cooling medium stream comprises at least a portion of the second gaseous nitrogen stream; a cooling apparatus operated under suitable conditions to produce the first cooled process gas stream, wherein the cooling apparatus comprises: a first heat exchanger or a mechanical means; a first inlet for providing the process gas stream; optionally, a second inlet for providing the first gaseous cooling medium; optionally, a first outlet for removal of a spent first gaseous cooling medium; and a second outlet for removal of the first cooled process gas stream; a first condenser vessel operated under suitable conditions to produce the first condensate stream, the second cooled process gas stream, and the first gaseous nitrogen stream, wherein the first condenser vessel comprises: a second heat exchanger; a third inlet for providing the first cooled process gas stream; a fourth inlet for providing the second cooling medium; a third outlet for removal of the first gaseous nitrogen stream; a fourth outlet for removal of the first condensate stream; and a fifth outlet for removal of the second cooled process gas stream; and a second condenser vessel operated under suitable conditions to produce the second condensate stream, the vent gas stream, and the second gaseous nitrogen stream, wherein the second condenser vessel comprises: a third heat exchanger; a sixth inlet for providing the second cooled process gas stream; a seventh inlet for providing the third cooling medium; a sixth outlet for removal of the vent gas stream; a seventh outlet for removal of the second gaseous nitrogen stream; and an eighth outlet for removal of the second condensate stream.

Other embodiments, including particular aspects of the embodiments summarized above, will be evident from the detailed description that follows.

### BRIEF DESCRIPTION OF THE DRAWINGS

The drawings described herein are for illustrative purposes only of selected embodiments and not all possible implementations, and are not intended to limit the scope of the present disclosure.

FIG. 1 illustrates a schematic of a system for recovering alkenes and nitrogen from process gas streams according to certain aspects of the present disclosure.

FIG. 2 illustrates a schematic of a system for recovering alkenes and nitrogen from process gas streams according to certain alternative aspects of the present disclosure.

FIG. 3 illustrates a schematic of a system for recovering alkenes and nitrogen from process gas streams according to certain alternative aspects of the present disclosure.

FIG. 4 illustrates a schematic of a system for recovering alkenes and nitrogen from process gas streams according to certain alternative aspects of the present disclosure.

FIG. 5 illustrates a schematic of a system for recovering alkenes and nitrogen from process gas streams according to certain alternative aspects of the present disclosure.

FIG. 6 illustrates a schematic of a system for recovering alkenes and nitrogen from process gas streams according to certain alternative aspects of the present disclosure.

Corresponding reference numerals indicate corresponding parts throughout the several views of the drawings.

### DETAILED DESCRIPTION OF THE INVENTION

In various aspects of the invention, methods and systems for recovering alkenes from process gas streams are provided.

### I. DEFINITIONS

The term “and/or” as used in a phrase such as “A and/or B” herein is intended to include “A and B”, “A or B”, “A”, and “B”.

As used herein, and unless otherwise specified, the term “Cₙ” means hydrocarbon(s) having n carbon atom(s) per molecule, wherein n is a positive integer. As used herein, and unless otherwise specified, the term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n.

As used herein, the term “alkene,” alternatively referred to as “olefin,” refers to a branched or unbranched unsaturated hydrocarbon having one or more carbon-carbon double bonds. A simple alkene comprises the general formula CₙH₂ₙ, where n is 2 or greater. Examples of alkenes include, but are not limited to ethene, propene, butene, pentene, hexene and heptene. “Alkene” is intended to embrace all structural isomeric forms of an alkene. For example, butene encompasses but-1-ene, (Z)-but-2-ene, etc.

As used herein, the term “oxygenate,” refers to oxygen-containing compounds having 1 to about 20 carbon atoms, 1 to about 10 carbon atoms, or 1 to about 4 carbon atoms. Exemplary oxygenates include alcohols, ethers, carbonyl compounds, e.g., aldehydes, ketones and carboxylic acids, and mixtures thereof. Particular non-limiting examples of oxygenates include methanol, ethanol, dimethyl ether, diethyl ether, methylethyl ether, di-isopropyl ether, dimethyl carbonate, dimethyl ketone, formaldehyde, acetaldehyde, acetic acid, and the like, and combinations thereof.

### II. METHODS FOR RECOVERING ALKENE AND NITROGEN FROM PROCESS GAS STREAMS

Methods for recovering alkene and nitrogen from process gas streams are provided herein. As discussed above, a process gas stream may comprise various impurities in addition to alkene gas. Thus, it is necessary to efficiently and effectively remove these impurities from the process gas stream in order to sufficiently recover alkenes and nitrogen. Furthermore, the process gas streams may be at higher temperatures. Therefore, the methods may further comprise initial cooling of the process gas stream in a cooling apparatus with a first gaseous cooling medium or by mechanical means under suitable conditions to produce a first cooled process gas stream.

The process gas stream comprises alkene gas, e.g., C₂-C₁₀ alkenes, C₂-C₈ alkenes or C₂-C₄ alkenes. In particular the process gas stream comprises ethylene and/or propylene. Additionally, the process gas stream may comprise alkene gas (e.g., C₂-C₄ alkenes), in an amount, based on the total weight of the process gas stream, of about 1.0 wt %, at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, at least about 99 wt % or about 99.5 wt %. It is understood herein that the amount of alkene gas provided herein corresponds to both a single alkene amount as well as combined amounts of alkenes, if one or more are present. For example, an alkene gas (e.g., C₂-C₄ alkenes) present in an amount of at least about 80 wt % encompasses a process gas stream comprising at least about 80 wt % ethylene as well as a process gas stream comprising at least about 80 wt % ethylene and propylene in combination. Additionally or alternatively, the process gas stream may comprise alkene gas (e.g., C₂-C₄ alkenes) in an amount, based on the total weight of the process gas stream, of about 1.0 wt % to about 99 wt %, about 1.0 wt % to about 90 wt %, about 1.0 wt % to about 80 wt %, about 1.0 wt % to about 60 wt %, about 1.0 wt % to about 40 wt %, about 1.0 wt % to about 20 wt %,%, about 20 wt % to about 99 wt %, about 40 wt % to about 99 wt %, about 60 wt % to about 99 wt %, about 70 wt % to about 99 wt %, about 80 wt % to about 99 wt %, about 80 wt % to about 95 wt %, about 80 wt % to about 90 wt %, or about 10 wt % to about 50 wt %.

A remaining portion, e.g., the balance, of the process gas stream may further comprise nitrogen and/or impurities, such as, but not limited to nitrogen, C₁-C₂₀ hydrocarbons (e.g., methane, ethane, propane, butane, butene, pentane, pentene, hexane, hexene, etc.), hydrogen, water and/or oxygenates (e.g., methyl acetate, ethyl acetate, vinyl acetate, methanol, dimethyl ether, acetaldehyde, etc.). In particular, the impurities may be selected from the group consisting of methane, ethane, propane, butane, pentane, pentene, hexane, hexene, hydrogen, methyl acetate, ethyl acetate, vinyl acetate, methanol, dimethyl ether, acetaldehyde, water, and a combination thereof. For example, the nitrogen and/or the impurities may be present in the process gas stream in an amount of at least about 1.0 wt %, at least about 5.0 wt %, at least about 10 wt %, at least about 15 wt %, at least bout 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt or about 99 wt %. Additionally or alternatively, the nitrogen and/or impurities may be present in the process gas stream in an amount of about 1.0 wt % to about 99 wt %, about 15 wt % to about 99 wt %, about 15 wt % to about 99 wt %, about 20 wt % to about 99 wt %, about 20 wt % to about 90 wt %, about 30 wt % to about 80 wt %, about 40 wt % to about 75 wt %, about 1.0 wt % to about 40 wt %, about 1.0 wt % to about 20, about 1.0 wt % to about 10 wt %, or about 1.0 wt % to about 5.0 wt. %. It is contemplated herein that the amount of impurities provided herein corresponds to both a single impurity amount as well as combined amounts of impurities, if one or more are present. For example, impurities present in an amount of at least about 10 wt % encompasses a process stream comprising at least about 10 wt % methanol as well as a process stream comprising at least about 10 wt % methanol and methane in combination.

In a particular embodiment, the process gas stream may comprise at least about 1.0 wt % nitrogen and less than about 80 wt % alkene gas (e.g., C₂-C₄ alkenes).

In various aspects, the process gas stream may enter the cooling apparatus at a temperature of at least about 0.0° C., at least about 10° C., at least about 15° C., at least about 20° C., at least about 30° C., at least about 40° C., at least about 50° C., at least about 60° C., at least about 70° C., at least about 80° C., at least about 90° C., or at least about 100° C. Additionally or alternatively, the process gas stream may enter the cooling apparatus at a temperature of about 0.0° C. to about 100° C., about 15° C. to about 100° C., about 15° C. to about 90° C., about 20° C. to about 80° C., or about 20° C. to about 70° C. Additionally, the process gas stream may enter the cooling apparatus at a pressure, optionally in combination with the above-described temperatures, of at least about 80 kPa, at least about 90 kPa, at least about 100 kPa, at least about 110 kPa, at least about 120 kPa, at least about 150 kPa, at least about 180 kPa, at least about 200 kPa, at least about 220 kPa, at least about 250 kPa, at least about 280 kPa, or about 300 kPa. For example, the process gas stream may enter the first condenser vessel at a pressure, optionally in combination with the above-described temperatures, of about 80 kPa to about 300 kPa, about 90 kPa to about 250 kPa, about 90 kPa to about 200 kPa, about 90 kPa to about 150 kPa or about 90 kPa to about 110 kPa. In particular, the process gas stream may enter the first condenser vessel at a temperature of at least about 15° C. and a pressure of at least about 90 kPa.

In order to produce the first cooled process gas stream, the process gas stream may be cooled by mechanical means or by circulating a first gaseous cooling medium through the cooling apparatus under suitable conditions. In various aspects, the cooling apparatus may comprise heat exchangers, such as a coil heat exchanger, a shell and tube heat exchanger and a plate heat exchanger. Suitable mechanical means include, but are not limited to a mechanical chiller, such as a reciprocating chiller, a centrifugal chiller and a screw driven chiller. Ethylene glycol and/or water may be used as the cooling medium in the mechanical chiller.

In the cooling apparatus, the process gas stream may be cooled to produce the first cooled process gas stream, which exits the cooling apparatus at a temperature of at least about −40° C., at least about −30° C., at least about −10° C., at least about 0.0° C., at least about 10° C., at least about 20° C., at least about 30° C., at least about 40° C., or about 50° C. In particular, the first cooled process gas stream may exit the cooling apparatus at temperature of at least about −40° C. Additionally or alternatively, the first cooled process gas stream may exit the cooling apparatus at a temperature of about −40° C. to about 50° C., about −20° C. to about 50° C., or about 0.0° C. to about 50° C. Additionally, the first cooled process gas stream may exit the cooling apparatus at a pressure, optionally in combination with the above-described temperatures, of at least about 80 kPa, at least about 90 kPa, at least about 100 kPa, at least about 110 kPa, at least about 120 kPa, at least about 150 kPa, at least about 180 kPa, at least about 200 kPa, at least about 220 kPa, at least about 250 kPa, at least about 280 kPa, or about 300 kPa. For example, the first cooled process gas stream may exit the cooling apparatus at a pressure, optionally in combination with the above-described temperatures, of about 80 kPa to about 300 kPa, about 90 kPa to about 250 kPa, about 90 kPa to about 200 kPa, about 90 kPa to about 150 kPa or about 90 kPa to about 110 kPa. In particular, the first cooled process gas stream may exit the cooling apparatus at a pressure at a temperature of at least about −40° C. and a pressure of at least about 90 kPa.

Suitable first gaseous cooling mediums include, but are not limited to gaseous nitrogen and/or gaseous air. The first gaseous cooling medium (e.g., gaseous nitrogen and/or gaseous air) may be provided and/or circulated in cooling apparatus at a temperature of at least about −196° C., at least about −190° C., at least about −180° C., at least about −170° C., at least about −160° C., at least about −150° C., at least about −140° C., at least about −130° C., at least about −120° C., at least about −110° C., at least about −100° C., at least about −90° C., at least about −80° C., at least about −70° C., at least about −60° C., at least about −50° C., at least about −40° C., at least about −30° C., at least about −20° C., at least about −10° C., or about 0.0° C. For example, the first gaseous cooling medium (e.g., gaseous nitrogen and/or gaseous air) may be provided and/or circulated in the cooling apparatus at a temperature of about −196° C. to about 0.0° C., about −196° C. to about −20° C., about −196° C. to about −40° C., about −170° C. to about −50° C., about −150° C. to about −50° C., about −120° C. to about −50° C., about −100° C. to about −50° C. or about −100° C. to about −70° C. Additionally, the first gaseous cooling medium (e.g., gaseous nitrogen and/or gaseous air) may be provided and/or circulated in the cooling apparatus, optionally in combination with the above-described temperatures, at a pressure of less than or equal to about 1600 kPa, less than or equal to about 1500 kPa, less than or equal to about 1300 kPa, less than or equal to about 1200 kPa, less than or equal to about 1000 kPa, less than or equal to about 800 kPa, less than or equal to about 700 kPa, less than or equal to about 500 kPa, less than or equal to about 300 kPa, or about 100 kPa. For example, the first gaseous cooling medium (e.g., gaseous nitrogen and/or gaseous air) may be provided and/or circulated in the cooling apparatus, optionally in combination with the above-described temperatures, at a pressure of about 100 kPa to about 1600 kPa, about 300 kPa to about 1500 kPa, about 500 kPa to about 1500 kPa, about 700 kPa to about 1300 kPa or about 800 kPa to about 1200 kPa. In particular, the first gaseous cooling medium (e.g., gaseous nitrogen) may be provided and/or circulated in the cooling apparatus at a temperature of at least about −100° C. and/or at a pressure of less than or equal to about 1500 kPa (e.g., about −90° C. and about 1000 kPa).

As the first gaseous medium (e.g., gaseous nitrogen) circulates and provides cooling through cooling apparatus, it can become heated and exit the cooling apparatus as a spent first gaseous medium at a higher temperature than the temperature of the first gaseous cooling medium. For example, the spent first gaseous medium may exit the cooling apparatus at a temperature of about −40° C. to about 40° C., about −30° C. to about 30° C., about −20° C. to about 10° C. or about −20° C. to about 0.0° C.

Following cooling of the process gas stream, in order to remove at least a portion of impurities present, the first cooled process gas stream may be cooled in a first condenser vessel with a second cooling medium under suitable conditions to produce a first condensate stream comprising at least a portion of the impurities and a second cooled process gas stream. The first condensate stream may be collected in a first condensate tank.

In order to produce the first condensate stream, the second cooling medium may be circulated through the first condenser vessel at a temperature suitable for condensing at least a portion of the impurities present in the process gas stream to produce the first condensate stream comprising at least a portion of the impurities and the second cooled process gas stream comprising alkenes (e.g., C₂-C₄ alkenes), which may exit the first condenser vessel. Thus, the second cooled process gas stream may have impurities present in an amount less than an amount of impurities present in the first cooled process gas stream entering the first condenser vessel. Further, the second cooled process gas stream exiting the first condenser vessel may have a temperature of less than or equal to about 0.0° C., less than or equal to about −10° C., less than or equal to about −20° C., less than or equal to about −30° C., less than or equal to about −40° C., less than or equal to about −50° C., less than or equal to about −60° C., less than or equal to about −70° C., less than or equal to about −80° C., less than or equal to about −90° C., less than or equal to about −100° C., less than or equal to about −110° C., less than or equal to about −120° C., less than or equal to about −130° C., less than or equal to about −140° C., or about −150° C. In particular, the second cooled process gas stream may have a temperature of less than or equal to about −10° C. Additionally or alternatively, the second cooled process gas stream may have a temperature of about −150° C. to about 0.0° C., about −150° C. to about −10° C., about −140° C. to about −20° C., about −130° C. to about −30° C., about −120° C. to about −40° C. or about −110° C. to about −50° C. Additionally or alternatively, the first condensate exiting the first condenser vessel may have a temperature of about −170° C. to about −104° C., about −170° C. to about −120° C., or about −170° C. to about −140° C., or about −150° C. to about −104° C.

In order to recover the alkenes (e.g., C₂-C₄ alkene) from the process gas stream, the process may further comprise cooling the second cooled process gas stream comprising alkenes (e.g., C₂-C₄ alkene) in a second condenser vessel under suitable conditions to produce a second condensate. A second cooling medium may be circulated through the second condenser vessel at a temperature suitable for condensing at least a portion of the alkene gas (e.g., C₂-C₄ alkenes) present in the process gas stream to produce the second condensate comprising alkenes (e.g., C₂-C₄ alkenes). The second condensate comprising alkenes (e.g., C₂-C₄ alkenes) may be collected in a second condensate tank. Additionally or alternatively, the second condensate exiting the second condenser vessel may have a temperature of about −170° C. to about −104° C., about −170° C. to about −120° C., or about −170° C. to about −140° C., or about −150° C. to about −104° C.

In various aspects, the second condensate may comprise at least about 40 wt %, at least about 50 wt %, at least about 60 wt % %, at least about 70 wt %, at least about 80 wt %, at least about 90 wt %, at least about 95 wt %, or about 99 wt % of the alkenes (e.g., C₂-C₄ alkenes), which were present in the process gas stream. For example, the second compensate may comprise about 40 wt % to about 99 wt %, about 50 wt % to about 99 wt %, about 70 wt % to about 99 wt %, about 80 wt % to about 99 wt %, or about 90 wt % to about 99 wt % of the alkenes (e.g., C₂-C₄ alkenes), which were present in the process gas stream, In particular, at least about 80 wt % f the alkenes (e.g., C₂-C₄ alkenes) present in the process gas stream may be present in the second condensate.

Examples of suitable first and second cooling mediums include, but are not limited to liquid nitrogen and/or gaseous nitrogen. As the first and second cooling medium (e.g., liquid and/or gaseous nitrogen) circulate and provide cooling through the first and second condenser vessels, respectively, they can become heated and exit the first and second condenser vessels as a first gaseous nitrogen stream and a second gaseous nitrogen stream, respectively. The first and/or second cooling medium (e.g., liquid and/or gaseous nitrogen) may be provided and/or circulated in the first and/or second condenser vessel at a temperature of at least about −200° C., at least about −196° C., at least about −190° C., at least about −180° C., at least about −170° C., at least about −160° C., or at least about −150° C. For example, the first and/or second cooling medium (e.g., liquid and/or gaseous nitrogen) may be provided and/or circulated in the first and/or second condenser vessel at a temperature of about −200° C. to about −150° C., about −196° C. to about −150° C., about −190° C. to about −150° C., or about −180° C. to about −160° C. Additionally, the first and/or second cooling medium (e.g., liquid nitrogen and/or gaseous nitrogen) may be provided and/or circulated in the first and/or second condenser vessel, optionally in combination with the above-described temperatures, at a pressure of less than or equal to about 1600 kPa, less than or equal to about 1500 kPa, less than or equal to about 1300 kPa, less than or equal to about 1200 kPa, less than or equal to about 1000 kPa, less than or equal to about 800 kPa, less than or equal to about 700 kPa, less than or equal to about 500 kPa, less than or equal to about 300 kPa, or about 100 kPa. For example, the first and/or second cooling medium (e.g., liquid and/or gaseous nitrogen) may be provided and/or circulated in the first and/or second condenser vessel, optionally in combination with the above-described temperatures, at a pressure of about 100 kPa to about 1600 kPa, about 300 kPa to about 1500 kPa, about 500 kPa to about 1500 kPa, about 700 kPa to about 1300 kPa or about 800 kPa to about 1200 kPa. In particular, the first and/or second cooling medium (e.g., liquid and/or gaseous nitrogen) may be provided and/or circulated in the first and/or second condenser vessel at a temperature of at least about −196° C. and/or at a pressure of less than or equal to about 1500 kPa (e.g., about −170° C. and about 1000 kPa). The first and/or second condenser vessel may each independently comprise heat exchangers, such as a coil heat exchanger, a shell and tube heat exchanger and a plate heat exchanger.

In various aspects, a vent gas (also referred to as a cleaned process gas stream) may also be produced in the second condenser vessel. The vent gas may comprise the non-condensable components of the process gas stream. For example, the vent gas may primarily comprise (e.g., ≥about 90 wt %, ≥about 95 wt %, ≥about 98 wt %, ≥about 99 wt %, or about 99.5 wt %) nitrogen and/or the impurities as described herein present in the process gas stream, such as but not limited to hydrogen and/or methane. In particular, the vent gas comprises nitrogen. Additionally or alternatively, the vent gas may comprise trace amounts (e.g., ≤5 about 5.0 wt %, ≤about 2.0 wt %) of alkenes (e.g., C₂-C₄ alkenes). Further, during the process, the vent gas may be continuously removed from the second condenser vessel or when the pressure in the second condenser vessel reaches a predetermined value. For example, the vent gas may be removed from the second condenser vessel when the pressure in the second condenser vessel reaches greater than about 100 kPa, at least about 200 kPa, at least about 300 kPa, at least about 400 kPa or about 500 kPa. Additionally or alternatively, the vent gas may be removed from the second condenser vessel when the pressure in the condenser vessel is about 100 kPa to about 500 kPa, about 200 kPa to about 400 kPa, or about 300 kPa to about 500 kPa. Further, the vent gas may exit the second condenser vessel at a temperature of about −170° C. to about −104° C., or about −160° C. to about −104° C. and/or at pressure of at least about 100 kPa, at least about 200 kPa, at least about 300 kPa, at least about 400 kPa, or at least about 500 kPa.

Additionally, process streams produced during the above-described methods may be advantageously recycled throughout to provide cooling during the process. For example, at least a portion of the first gaseous nitrogen stream and/or the second gaseous nitrogen stream may be recycled to the cooling apparatus for use as at least a portion of the first gaseous cooling medium. Additionally or alternatively, at least a portion of the second gaseous stream may be recycled to the first condenser vessel for use as at least a portion of the second cooling medium, for example, when the mechanical means are used for cooling the process gas stream. In some aspects, the second cooling medium may comprise at least a portion of the second gaseous nitrogen stream and optionally, liquid nitrogen. In a further embodiment, all cooling during the method may be provided by the various gaseous nitrogen streams (e.g., first gaseous cooling medium, first gaseous nitrogen stream, second gaseous nitrogen stream) and optionally supplemented with liquid nitrogen. In another embodiment, cooling during the method is not provided via mechanical means. In another embodiment, all cooling during the method may be provided by the gaseous nitrogen streams produced during the process, e.g., by utilizing the first gaseous nitrogen stream and/or second gaseous nitrogen stream, and optionally supplemented with liquid nitrogen.

The methods may further comprise maintaining the pressure of the various gaseous nitrogen streams (e.g., first gaseous cooling medium, first gaseous nitrogen stream, second gaseous nitrogen stream) so that they may more easily be transported throughout a plant. For example, the pressure of the various gaseous nitrogen streams (e.g., first gaseous cooling medium, first gaseous nitrogen stream, second gaseous nitrogen stream) may be maintained at least about 100 kPa, at least about 250 kPa, at least about 500 kPa, at least about 1000 kPa, at least about 1500 kPa or about 2000 kPa. Additionally or alternatively, the pressure of the various gaseous nitrogen streams (e.g., first gaseous cooling medium, first gaseous nitrogen stream, second gaseous nitrogen stream) may be maintained at about 100 kPa to about 2000 kPa, about 250 kPa to about 2000 kPa or about 250 kPa to about 1500 kPa. In particular, the pressure of the first gaseous cooling medium within the first cooling apparatus may be maintained at a pressure of at least about 500 kPa. Maintaining the pressure of the various gaseous nitrogen streams (e.g., first gaseous cooling medium, first gaseous nitrogen stream, second gaseous nitrogen stream) may be achieved by any suitable means known in the art. For example, a back pressure regulator and/or a blower could be used with the various nitrogen streams.

A. Optional Additional Steps

Optionally, the method may further comprise pressurizing the second condensate stream. For example, the method may comprise halting flow of the process gas stream and optionally, the first gaseous cooling medium to the cooling apparatus, flow of the first cooled process gas stream and the second cooling medium to the first condenser vessel, flow of the first condensate to the first condensate tank, flow of the second cooled process gas stream and the third cooling medium to the second condenser vessel and/or flow of the second condensate to the second condensate tank. Optionally, the process gas stream may be directed to another cooling apparatus and/or condenser vessel in series where it may undergo cooling as described herein. Further, heat may be applied to the second condensate tank to produce pressurized liquid alkene (e.g., C₂-C₄ alkene) and an alkene (e.g., C₂-C₄ alkene) vent gas. The heat provided to the at least one condensate tank may vaporize at least a portion of the alkenes (e.g., C₂-C₄ alkene) in the first condensate to produce pressurized liquid alkene (e.g., C₂-C₄ alkene) and an alkene (e.g., C₂-C₄ alkene) vent gas. The alkene (e.g., C₂-C₄ alkene) vent gas may also comprise other components, such as methane. Any suitable means for providing heat to the second condensate tank may be used, for example, heat may be provided via a heater (e.g., electric heater), via heated gaseous nitrogen, via heated air or via an ambient vaporizer. Further, the heat may be provided at a suitable temperature for a suitable amount of time to produce pressurized liquid alkene (e.g., C₂-C₄ alkene) at a desirable pressure as determined by the needs of the process, for example, at a pressure of about 100 kPa to about 1500 kPa, about 200 kPa to about 800 kPa or about 300 kPa to about 500 kPa. For example, the pressurized liquid alkene (e.g., C₂-C₄ alkene) may be maintained at a temperature of at least about 100 kPa. Additionally or alternatively, the pressurized liquid alkene (e.g., C₂-C₄ alkene) may have a temperature of less than about −104° C., e.g., about −170° C. to about −104° C., about −170° C. to about −120° C., about −170° C. to about −140° C., or about −150° C. to about −104° C. The pressurized liquid alkene (e.g., C₂-C₄ alkene) may then be recycled and reused as needed in the process.

The methods described herein may further comprise a defrost cycle to melt any frozen alkenes and/or impurities in the condenser vessels (e.g., first condenser vessel, second condenser vessel). The defrost cycle may be performed as needed by the process. For example, the defrost cycle may be commenced when the pressure drop within the condenser vessels (e.g., first condenser vessel, second condenser vessel) increases to an undesirable level and/or when the level of condensate in the condensate tanks (e.g., first condensate tank, second condensate tank) is too high. The defrost cycle may comprise halting flow of the process gas stream and optionally, the first gaseous cooling medium to the cooling apparatus, flow of the first cooled process gas stream and the second cooling medium to the first condenser vessel, flow of the first condensate to the first condensate tank, flow of the second cooled process gas stream and the third cooling medium to the second condenser vessel and/or flow of the second condensate to the second condensate tank. Then the condenser vessels (e.g., first condenser vessel, second condenser vessel) may be heated to produce at least a third condensate stream comprising alkenes (e.g., C₂-C₄ alkene). The heating of the condenser vessels (e.g., first condenser vessel, second condenser vessel) may be provided by any suitable means for defrosting the condenser vessel. For example, heating may be provided by gaseous nitrogen. The gaseous nitrogen may be heated to a suitable temperature (e.g., greater than about −104° C. up to about 25° C.) prior to introduction into the condenser vessels (e.g., first condenser vessel, second condenser vessel). The defrost cycle may further comprise draining the third condensate stream from the second condenser vessel to collect in the second condensate tank or in a third condensate tank.

### III. SYSTEMS FOR RECOVERING ALKENE AND NITROGEN FROM PROCESS GAS STREAMS

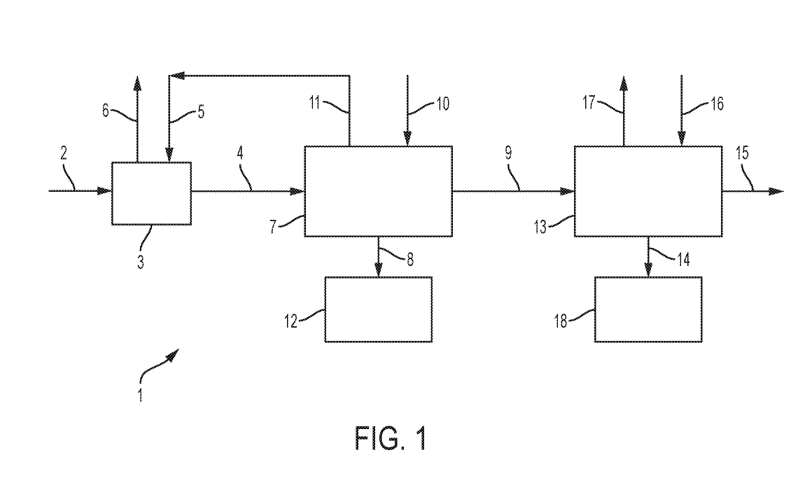

Systems for recovering alkene (e.g., C₂-C₄ alkene) and nitrogen from a process gas stream as described herein are also provided. Referring to FIG. 1, the system 1 may comprise a process gas stream 2 (e.g., having a temperature of at least about 15° C.) comprising alkenes (e.g., C₂-C₄ alkene) and impurities, which is provided to a cooling apparatus 3 via a first inlet (not shown) wherein the process gas stream is cooled to produce a first cooled process gas stream 4. In particular, the process gas stream 2 may comprise alkenes (e.g., C₂-C₄ alkene) as described herein and impurities (e.g., nitrogen, hydrogen, water, C₁-C₁₀ hydrocarbons, oxygenates) as described herein. In certain aspects, the alkenes are ethylene and/or propylene.

In order to produce the first cooled process gas stream 4 (e.g., having a temperature of at least about −40° C.), the system 1 may optionally further comprise a first gaseous cooling medium stream 5 (e.g., gaseous nitrogen) as described herein provided via a second inlet (not shown), which may circulated through cooling apparatus 3 at a temperature suitable for cooling the process gas stream 2. As the first gaseous cooling medium stream 5 (e.g., gaseous nitrogen) circulates through the cooling apparatus, it may be heated and exit the system 1 as a spent first gaseous medium 6 via a first outlet (not shown). In various aspects, the cooling apparatus 3 may comprise, consist essentially of, or consist of a first heat exchanger (e.g., shell and tube heat exchanger, plate heat exchanger, coil heat exchanger). Alternatively, the process gas stream 2 may be cooled via mechanical means (shown in later figures). Suitable mechanical means include, but are not limited to a mechanical chiller, such as a water chiller.

Following cooling of the process gas stream, the first cooled process gas stream 4 may be removed from the cooling apparatus 3 via a second outlet (not shown) and provided to a first condenser vessel 7 via a third inlet (not shown), wherein a first condensate stream 8 and a second cooled process gas stream 9 are produced.

In order to produce the first condensate stream 8 comprising at least a portion of the impurities as described herein and the second cooled process gas stream 9 as described herein, the system 1 may further comprise a second cooling medium stream 10 provided via a fourth inlet (not shown), which may be circulated through the first condenser vessel 7, at a temperature suitable for condensing at least a portion of the impurities present in the first cooled process gas stream 4 to produce the first condensate stream 8 comprising impurities. The first condenser vessel 7 may comprise, consist essentially of, or consist of a second heat exchanger (e.g., shell and tube heat exchanger, plate heat exchanger, coil heat exchanger). The second cooling medium stream 10 may comprise a suitable cooling medium as described herein, for example, liquid nitrogen and/or gaseous nitrogen. As the second cooling medium stream 10 (e.g., gaseous and/or liquid nitrogen) circulates through the first condenser vessel 7, it may be heated and exit the system 1 as a first gaseous nitrogen stream 11 via a third outlet (not shown).

A first condensate tank 12 may be present in the system 1 for collection of the first condensate stream 8, which may be removed via a fourth outlet (not shown) in the first condenser vessel 7, for example, as the first condensate stream 8 drains from the first condenser vessel 7. The first condensate tank 12 may comprise a fifth inlet (not shown) for receiving the first condensate stream 8.

The system 1 may further comprise a second condenser vessel 13 for alkene (e.g., C₂-C₄ alkene) recovery. In particular, the second cooled process gas stream 9 may exit the first condenser vessel 7 via a fifth outlet (not shown) and enter the second condenser vessel 13 via a sixth inlet (not shown). The second condenser vessel 13 may be operated under suitable conditions to produce a second condensate stream 14 comprising alkenes (e.g., C₂-C₄ alkene) and a vent gas stream 15 as described herein (also referred to as a cleaned process gas stream). The vent gas stream 15 may exit the second condenser vessel 13 via a sixth outlet (not shown) and may primarily comprise (e.g., ≥about 90 wt %, ≥about 95 wt %, ≥about 98 wt %, %, ≥about 99 wt %, or about 99.5 wt %) the non-condensable components of the second process gas stream 9, e.g., hydrogen, nitrogen and/or methane. In particular, the vent gas stream 15 comprises nitrogen.

In order to produce the second condensate stream 14 as described herein and the vent gas stream 15 as described herein, the system 1 may further comprise a third cooling medium stream 16 provided via a seventh inlet (not shown), which may be circulated through the second condenser vessel 13, at a temperature suitable for condensing at least a portion of the alkenes (e.g., C₂-C₄ alkene) present in the second cooled process gas stream 9 to produce the second condensate stream 14 comprising alkenes (e.g., C₂-C₄ alkene). The second condenser vessel 13 may comprise, consist essentially of, or consist of a second heat exchanger (e.g., shell and tube heat exchanger, plate heat exchanger, coil heat exchanger). The third cooling medium stream 16 may comprise a suitable cooling medium as described herein, for example, liquid nitrogen and/or gaseous nitrogen. As the third cooling medium stream 16 (e.g., gaseous and/or liquid nitrogen) circulates through the second condenser vessel 13, it may be heated and exit the system 1 as a second gaseous nitrogen stream 17 via a seventh outlet (not shown).

A second condensate tank 18 may be present in the system 1 for collection of the second condensate stream 14, which may be removed via an eighth outlet (not shown) in the second condenser vessel 13. The second condensate tank 18 may comprise an eighth inlet (not shown) for receiving the second condensate stream 14.

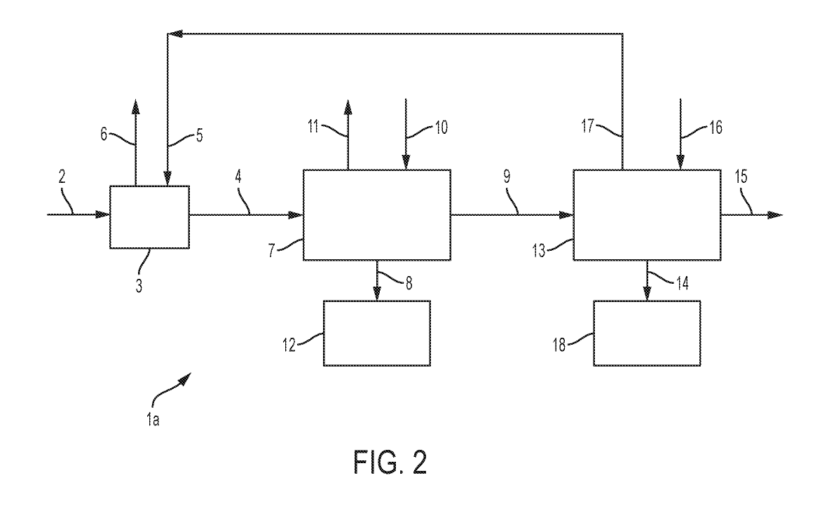

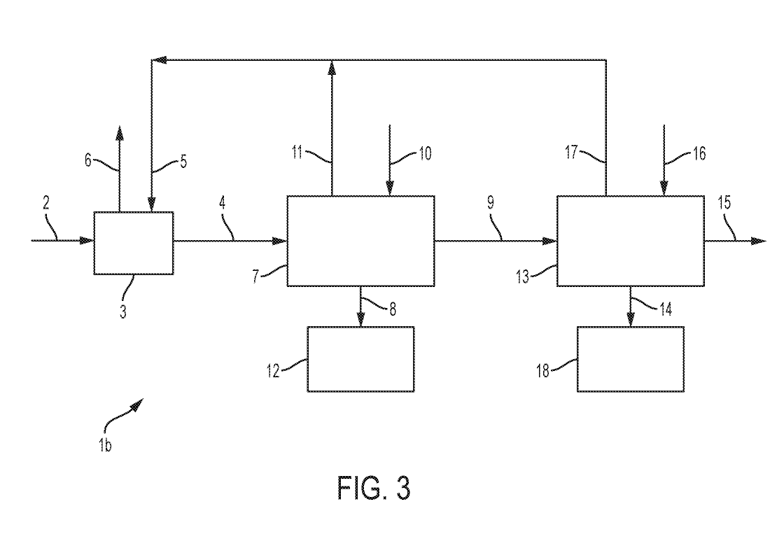

Advantageously, as shown in FIG. 1, at least a portion of the first gaseous nitrogen stream 11 may be recycled and used as at least a portion of the first gaseous cooling medium stream 5. Alternatively, as shown in FIG. 2, at least a portion of the second gaseous nitrogen stream 17 may be recycled and used as at least a portion of the first gaseous cooling medium stream 5. In another alternative embodiment, as shown in FIG. 3, at least a portion of both the first gaseous nitrogen stream 11 and the second gaseous nitrogen stream 17 may be recycled and used as at least a portion of the first gaseous cooling medium stream 5. It is contemplated herein, that the first gaseous cooling medium stream 5, for example gaseous nitrogen, may be provided from an initial gaseous nitrogen source at the beginning of the processed described herein and then once the first gaseous nitrogen stream 11 and/or the second gaseous nitrogen stream 17 are produced, those streams may be recycled for use as the first gaseous cooling medium stream 5. Additionally, it is contemplated herein that once the first gaseous nitrogen stream 11 and/or the second gaseous nitrogen stream 17 are recycled for use as the first gaseous cooling medium stream 5, further gaseous nitrogen may be provided to the first gaseous cooling medium stream 5 from the initial gaseous nitrogen source as well, if needed.

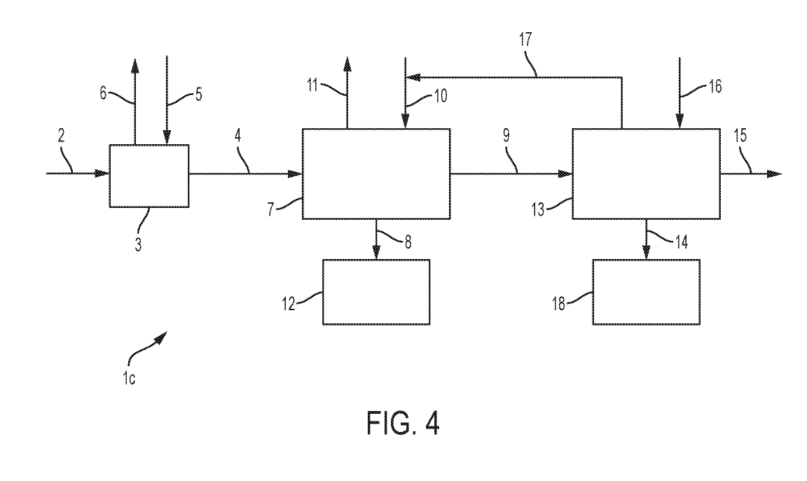

In still another alternative embodiment, as shown in FIG. 4, at least a portion of the second gaseous nitrogen stream 17 may be recycled for use as at least a portion of the second cooling medium stream 10. It is understood herein, that the second cooling medium stream 10, for example liquid nitrogen, may be provided from an initial liquid nitrogen source at the beginning of the processed described herein and then once the second gaseous nitrogen stream 17 is produced, it may be recycled for use as at least a portion of the second cooling medium stream 10. Additionally, it is contemplated herein, that once the second gaseous nitrogen stream 17 is recycled for use as the second cooling medium stream 10, further liquid nitrogen may be provided to the second cooling medium stream 10 from the initial liquid nitrogen source as well, if needed. Thus, in various aspects, the second cooling medium stream 10 may comprise the second gaseous nitrogen stream 17 and optionally, liquid nitrogen.

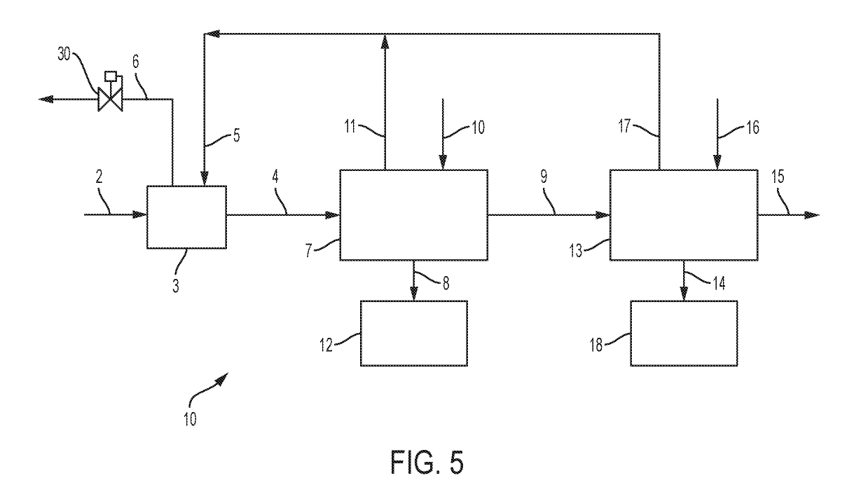

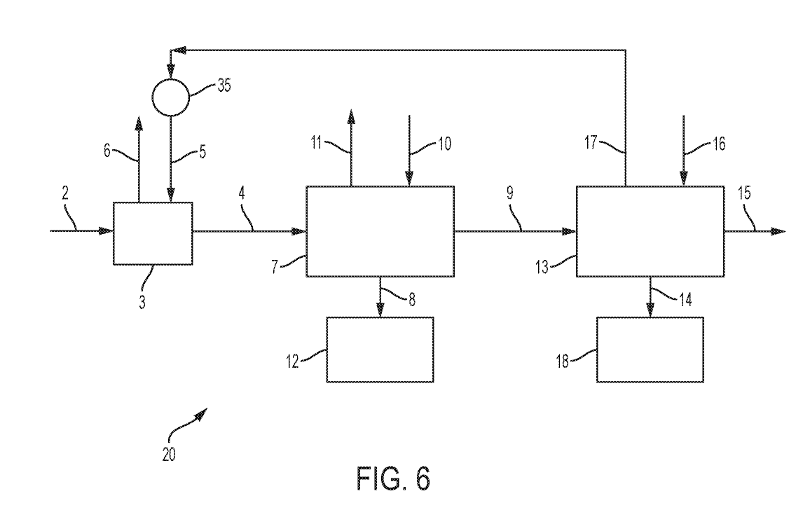

The systems described herein (e.g., FIGS. 1-4) may further comprise means for maintaining the pressure of the various gaseous nitrogen streams (e.g., first gaseous cooling medium stream 5, first gaseous nitrogen stream 11, second gaseous nitrogen stream 17) so that they may more easily be transported throughout a plant. For example, as described herein, the pressure of the various gaseous nitrogen streams (e.g., first gaseous cooling medium, first gaseous nitrogen stream, second gaseous nitrogen stream) may be maintained at least about 500 kPa. Suitable means for maintaining the pressure of the various gaseous nitrogen streams (e.g., first gaseous cooling medium stream 5, first gaseous nitrogen stream 11, second gaseous nitrogen stream 17) can include, but are not limited to a back pressure regulator and/or a blower. For example, as shown in FIG. 5, a back pressure regulator 30 can be included in a system 10 on the spent first gaseous medium stream 6. It is contemplated herein that the back pressure regulator 30 may be present on other streams, as needed. Alternatively, as shown in FIG. 6, a blower 35 can be included in a system 20 on the first gaseous cooling medium stream 5. It is contemplated herein that the blower 35 may be present on other streams, as needed, for example, the spent first gaseous medium stream 6.

### IV. Further Embodiments

The invention can additionally or alternatively include one or more of the following embodiments.

### Embodiment 1

A method for recovering C₂-C₄ alkene (e.g., ethylene and/or propylene) and nitrogen from a process gas stream comprising: cooling the process gas stream comprising C₂-C₄ alkene (e.g., ethylene and/or propylene) in a cooling apparatus with a first gaseous cooling medium (e.g., gaseous nitrogen) or by mechanical means under suitable conditions to produce a first cooled process gas stream; cooling the first cooled process gas stream in a first condenser vessel with a second cooling medium (e.g., liquid and/or gaseous nitrogen) under suitable conditions to produce a first condensate comprising impurities (e.g., hydrogen, water, C₁-C₂₀ hydrocarbons, and/or oxygenates), a second cooled process gas stream and a first gaseous nitrogen stream; cooling the second cooled process gas stream in a second condenser vessel with a third cooling medium (e.g., liquid and/or gaseous nitrogen) under suitable conditions to produce a second condensate comprising C₂-C₄ alkene (e.g., ethylene and/or propylene), a vent gas stream (e.g., nitrogen) and a second gaseous nitrogen stream; and recycling at least one of the following: (i) at least a portion of the first gaseous nitrogen stream and/or the second gaseous nitrogen stream to the cooling apparatus for use as at least a portion of the first gaseous cooling medium (e.g., gaseous nitrogen); (ii) at least a portion of the second gaseous nitrogen stream to the first condenser vessel for use as at least a portion of the second cooling medium (e.g., liquid and/or gaseous nitrogen).

### Embodiment 2

The method of embodiment 1, further comprising maintaining the pressure of the first gaseous cooling medium within the first cooling apparatus at least about 500 kPa.

### Embodiment 3

The method of embodiment 1 or 2, wherein the process gas stream enters the cooling apparatus at a temperature of at least about 15° C. and a pressure of at least about 90 kPa and/or the first cooled process gas stream exits the cooling apparatus at a temperature of at least about −40° C.

### Embodiment 4

The method of any one of the previous embodiments, wherein the mechanical means is selected from the group consisting of a mechanical chiller and/or the cooling apparatus, the first condenser vessel and/or wherein the second condenser vessel each independently comprise a coil heat exchanger, a shell and tube heat exchanger or a plate heat exchanger.

### Embodiment 5

The method of any one of the previous embodiments, wherein the first gaseous cooling medium is provided at a temperature of at least about −100° C. and a pressure of less than or equal to about 1500 kPa and/or wherein the second cooling medium and/or the third cooling medium is provided at a temperature at least about −196° C. and a pressure of less than or equal to about 1500 kPa.

### Embodiment 6

The method of any one of the previous embodiments, wherein the second cooling medium comprises at least a portion of the second gaseous nitrogen stream and optionally, liquid nitrogen.

### Embodiment 7

A system for recovering C₂-C₄ alkene (e.g., ethylene and/or propylene) from a process gas stream comprising: a process gas stream comprising C₂-C₄ alkene (e.g., ethylene and/or propylene); optionally, a first gaseous cooling medium stream (e.g., gaseous nitrogen); a first cooled process gas stream; a second cooling medium stream (e.g., liquid and/or gaseous nitrogen); a first condensate stream comprising impurities (e.g., hydrogen, water, C₁-C₂₀ hydrocarbons, and/or oxygenates); a first gaseous nitrogen stream; a second cooled process gas stream; a third cooling medium stream (e.g., liquid and/or gaseous nitrogen); a second condensate stream comprising C₂-C₄ alkene (e.g., ethylene and/or propylene); a vent gas stream; and a second gaseous nitrogen stream, wherein at least one of the following is present: (i) the first gaseous cooling medium stream (e.g., gas nitrogen), if present, comprises at least a portion of the first gaseous nitrogen stream and/or the second gaseous nitrogen stream; or (ii) the second cooling medium stream (e.g., liquid and/or gaseous nitrogen) comprises at least a portion of the second gaseous nitrogen stream; a cooling apparatus operated under suitable conditions to produce the first cooled process gas stream, wherein the cooling apparatus comprises: a first heat exchanger or a mechanical means; a first inlet for providing the process gas stream; optionally, a second inlet for providing the first gaseous cooling medium; optionally, a first outlet for removal of a spent first gaseous cooling medium; and a second outlet for removal of the first cooled process gas stream; a first condenser vessel operated under suitable conditions to produce the first condensate stream, the second cooled process gas stream, and the first gaseous nitrogen stream, wherein the first condenser vessel comprises: a second heat exchanger; a third inlet for providing the first cooled process gas stream; a fourth inlet for providing the second cooling medium; a third outlet for removal of the first gaseous nitrogen stream; a fourth outlet for removal of the first condensate stream; and a fifth outlet for removal of the second cooled process gas stream; and a second condenser vessel operated under suitable conditions to produce the second condensate stream, the vent gas stream, and the second gaseous nitrogen stream, wherein the second condenser vessel comprises: a third heat exchanger; a sixth inlet for providing the second cooled process gas stream; a seventh inlet for providing the third cooling medium; a sixth outlet for removal of the vent gas stream; a seventh outlet for removal of the second gaseous nitrogen stream; and an eighth outlet for removal of the second condensate stream.

### Embodiment 8

The system of embodiment 7 further comprising a means for maintaining the pressure of the first gaseous cooling medium within the first cooling apparatus at least about 500 kPa.

### Embodiment 9

The system of embodiment 7 or 8, wherein the first heat exchanger, second heat exchanger and/or the third heat exchanger each independently comprise a coil heat exchanger, a shell and tube heat exchanger or a plate heat exchanger.

### Embodiment 10

They system of embodiment 7 to 9, wherein the second cooling medium comprises at least a portion of the second gaseous nitrogen stream and optionally, liquid nitrogen.

### EXAMPLES

### Example 1

Ethylene recovery from a process gas in a system similar to the configuration shown in FIG. 2 was simulated using the commercialized simulation software, UniSim Design R440. In the simulation, the second gaseous nitrogen stream produced in a second condenser vessel (ethylene condenser) was recycled back to the cooling apparatus (gas/gas heat exchanger) for use as the first gaseous cooling medium. The process gas comprised 30 wt % ethylene, 10 wt % butane, and 60 wt % nitrogen. The details of each of the streams during the simulation are provided below in Table 1.

TABLE 1

| | Flowrate | Temperature | Pressure |
|---|---|---|---|
| Stream | (kg/hr) | (° C.) | (kPa) |
| Process Gas | ~500 | ~25 | ~100 |
| First Gaseous Cooling | ~464 | ~−90 | ~1000 |
| Medium/Second Gaseous Nitrogen | | | |
| Spent First Gaseous Cooling | ~464 | ~−9 | ~1000 |
| Medium | | | |
| First Cooled Process Gas | ~500 | ~−40 | ~100 |
| Second Cooling Medium (Liquid | ~164 | ~−170 | ~1000 |
| Nitrogen) | | | |
| First Gaseous Nitrogen | ~164 | ~−90 | ~1000 |
| First Condensate (Liquid | ~40 | ~−70 | ~100 |
| impurities) | | | |
| Second Cooled Process Gas | ~460 | ~−70 | ~100 |
| Third Cooling Medium (Liquid | ~464 | ~−170 | ~100 |
| Nitrogen) | | | |
| Vent Gas/Cleaned Process Gas | ~305 | ~−150 | ~100 |
| Second Condensate (Liquid | ~155 | ~−150 | ~100 |
| Ethylene) | | | |

As shown in Table 1, the simulation predicted that the second gaseous nitrogen stream exiting the second condenser (an ethylene condenser), was sufficient to cool the process gas stream in the cooling apparatus from ˜25° C. to ˜−40° C. without hindering a 10° C. temperature approach.

What is claimed is:

1. A method for recovering C₂-C₄ alkene and nitrogen from a process gas stream comprising:
cooling the process gas stream comprising C₂-C₄ alkene in a cooling apparatus with a first gaseous cooling medium or by mechanical means under suitable conditions to produce a first cooled process gas stream;
cooling the first cooled process gas stream in a first condenser vessel with a second cooling medium under suitable conditions to produce a first condensate comprising impurities, a second cooled process gas stream and a first gaseous nitrogen stream;
cooling the second cooled process gas stream in a second condenser vessel with a third cooling medium under suitable conditions to produce a second condensate comprising C₂-C₄ alkene, a vent gas stream and a second gaseous nitrogen stream; and
recycling at least one of the following:
(i) at least a portion of the first gaseous nitrogen stream and/or the second gaseous nitrogen stream to the cooling apparatus for use as at least a portion of the first gaseous cooling medium; or
(ii) at least a portion of the second gaseous nitrogen stream to the first condenser vessel for use as at least a portion of the second cooling medium.

2. The method of claim 1 further comprising maintaining the pressure of the first gaseous cooling medium within the first cooling apparatus at a pressure of at least about 500 kPa.

3. The method of claim 1, wherein the process gas stream enters the cooling apparatus at a temperature of at least about 15° C. and a pressure of at least about 90 kPa.

4. The method of claim 1, wherein the first cooled process gas stream exits the cooling apparatus at a temperature of at least about −40° C.

5. The method of claim 1, wherein the impurities are water, hydrogen, C₁-C₂₀ hydrocarbons, and/or oxygenates.

6. The method of claim 1, wherein the cooling apparatus, the first condenser vessel and/or the second condenser vessel each independently comprise a coil heat exchanger, a shell and tube heat exchanger or a plate heat exchanger.

7. The method of claim 1, wherein the second cooling medium and/or the third cooling medium comprises liquid nitrogen and/or gaseous nitrogen.

8. The method of claim 1, wherein the first gaseous cooling medium is provided at a temperature of at least about −100° C. and a pressure of less than or equal to about 1500 kPa.

9. The method of claim 1, wherein the second cooling medium and/or the third cooling medium is provided at a temperature at least about −196° C. and a pressure of less than or equal to about 1500 kPa.

10. The method of claim 1, wherein the second cooling medium comprises at least a portion of the second gaseous nitrogen stream and optionally, liquid nitrogen.

11. The method of claim 1, wherein the C₂-C₄ alkene is ethylene and/or propylene.

12. A system for recovering C₂-C₄ alkene from a process gas stream comprising:
a process gas stream comprising C₂-C₄ alkene;
optionally, a first gaseous cooling medium stream;
a first cooled process gas stream;
a second cooling medium stream;
a first condensate stream comprising impurities;
a first gaseous nitrogen stream;
a second cooled process gas stream;
a third cooling medium stream;
a second condensate stream comprising C₂-C₄ alkene;
a vent gas stream; and
a second gaseous nitrogen stream, wherein at least one of the following is present:
(i) the first gaseous cooling medium stream, if present, comprises at least a portion of the first gaseous nitrogen stream and/or the second gaseous nitrogen stream; or
(ii) the second cooling medium comprises at least a portion of the second gaseous nitrogen stream;
a cooling apparatus operated under suitable conditions to produce the first cooled process gas stream, wherein the cooling apparatus comprises:
a first heat exchanger or a mechanical means;
a first inlet for providing the process gas stream;
optionally, a second inlet for providing the first gaseous cooling medium;
optionally, a first outlet for removal of a spent first gaseous cooling medium; and
a second outlet for removal of the first cooled process gas stream;
a first condenser vessel operated under suitable conditions to produce the first condensate stream, the second cooled process gas stream, and the first gaseous nitrogen stream, wherein the first condenser vessel comprises:
a second heat exchanger;
a third inlet for providing the first cooled process gas stream;
a fourth inlet for providing the second cooling medium;
a third outlet for removal of the first gaseous nitrogen stream;
a fourth outlet for removal of the first condensate stream; and
a fifth outlet for removal of the second cooled process gas stream; and
a second condenser vessel operated under suitable conditions to produce the second condensate stream, the vent gas stream, and the second gaseous nitrogen stream, wherein the second condenser vessel comprises:
a third heat exchanger;
a sixth inlet for providing the second cooled process gas stream;
a seventh inlet for providing the third cooling medium;
a sixth outlet for removal of the vent gas stream;
a seventh outlet for removal of the second gaseous nitrogen stream; and
an eighth outlet for removal of the second condensate stream.

13. The system of claim 12 further comprising a means for maintaining the pressure of the first gaseous cooling medium within the first cooling apparatus at least about 500 kPa.

14. The system of claim 12, wherein the impurities are hydrogen, water, C₁-C₂₀ hydrocarbons, and/or oxygenates.

15. The system of claim 12, wherein the first heat exchanger, second heat exchanger and/or the third heat exchanger each independently comprise a coil heat exchanger, a shell and tube heat exchanger or a plate heat exchanger.

16. The system of claim 12, wherein the second cooling medium comprises at least a portion of the second gaseous nitrogen stream and optionally, liquid nitrogen.

17. The system of claim 12, wherein the second cooling medium and/or third cooling medium comprises liquid nitrogen and/or gaseous nitrogen.

18. The system of claim 12, wherein the C₂-C₄ alkene is ethylene and/or propylene.
